(19) United States
(12) Patent Application Publication
(10) Pub. No.: US 2010/0296969 A1
(43) Pub. Date: Nov. 25, 2010

(54) PROCESS FOR STERILIZING ACELLULAR SOFT TISSUE UNDER VACUUM

(75) Inventors: Manh-Dan Ngo, Rochester, NY (US); Jeffrey Stuart Cartmell, Freehold, NJ (US); Arthur Gertzman, Flemington, NJ (US)

Correspondence Address: FROMMER LAWRENCE & HAUG, 745 FIFTH AVENUE- 10TH FL., NEW YORK, NY, 10151, US

(73) Assignee: Musculoskeletal Transplant Foundation, Edison, NJ (US)

(21) Appl. No.: 12/664,296

(22) PCT Filed: Jun. 12, 2008
(86) PCT No.: PCT/US2008/066697

§ 371 Date: Aug. 5, 2010

Related U.S. Application Data
(60) Provisional application No. 60/929,085, filed on Jun. 12, 2007.

(51) Int. Cl. A61L 2/20

(52) U.S. Cl. 422/33

(57) ABSTRACT

The present invention is a process for preparing skin removed from a human donor, removal of cellular components and sterilizing the decellularized skin. The process comprises the following steps:
(a) decellularizing the skin including soaking the skin in a detergent and rinsing same with sterile water;
(b) sterilizing the skin under vacuum with one or more of ozone, vapor H₂O₂, plasma H₂O₂, ethylene oxide gas, or sodium hydroxide for a time period to achieve a concentration to achieve sterilization of the skin; and
(c) processing the skin by cutting the skin to a designated size.

### RELATED APPLICATIONS

This application claims the benefit of priority of U.S. Provisional Application No. 60/929,085 filed Jun. 12, 2007.

The foregoing applications, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF INVENTION

The present invention is generally directed toward methods of treatment of soft tissue including decellularizing and sterilization of the soft tissue by placing the same under vacuum and treating the soft tissue with an oxide or allotropic form of oxygen for a period of time for implantation into another human being.

### BACKGROUND OF THE INVENTION

Techniques for restoring structure and function to damaged tissue are used routinely in the area of reconstructive surgery. Tissue transplantation is another way of restoring function by replacing, regenerating, repairing, rebuilding or protecting the damaged tissue. However, problems exist when there is a transfer of biological material from one individual to another. Tissue rejection is a significant risk associated with transplantation, even with a good histocompatability match. Immunosuppressive drugs such as cyclosporin and FK506 are usually given to the patient to prevent rejection. These immunosuppressive drugs however, have a narrow therapeutic window between adequate immunosuppression and toxicity. Prolonged immunosuppression can weaken the immune system, which can lead to a threat of infection.

The present invention is directed toward a process for use in the preparation of acellular, i.e. (essentially lacking in living cells and/or non-living cells,) soft-tissue implants derived from tissue products taken from mammals and in particular the skin of human donors. The decellularized grafts produced are significantly improved in long-term durability and function when used in clinical applications.

Various methods have been used in the treatment of soft tissue and such representative methods are disclosed in a number of prior art patents and publications.

The advantages of retaining an acellular matrix, composed primarily of a collagenous component, have been explored in WO 84/0488 for the production of sterile body implants. In this method, a variety of tissues were extracted sequentially with non-ionic and ionic detergents to yield structures essentially free of cellular membranes, nucleic acids, lipids and cytoplasmic components. The treatment consists of sequential extractions with a non-denaturing detergent and a denaturing detergent to form an acellular matrix of collagen.

U.S. Pat. No. 4,776,853 issued Oct. 11, 1988 is directed toward a process for preparing biological material for implant in a mammal's cardiovascular system, respiratory system or soft tissue. The process comprises: (1) isolating a desired tissue sample of the biological material from a donor; (2) extracting the tissue sample with an hypotonic buffer solution at a mild alkaline pH, the buffer solution including active amounts of proteolytic inhibitors and antibiotics; (3) extracting the tissue sample with a buffered solution having a high concentration of salt, the solution being at a mild alkaline pH and including a non-ionic detergent with protease inhibitors and antibiotics; (4) subjecting the tissue sample to enzymatic digestion in a buffered saline solution, the enzymes consisting of purified protease-free dioxyribonuclease and ribonuclease; (5) extracting the tissue sample with an anionic detergent at a mild alkaline pH; and (6) storing the tissue sample in physiologic buffered solutions.

Another soft tissue process is shown in U.S. Pat. No. 6,734,018 issued May 11, 2004 which is directed toward a process for preparing an acellular soft tissue graft for implantation into a mammalian system. The process extracts a soft tissue sample with an extracting solution including one or more nonionic detergents and one or more endonucleases, to produce extracted tissue and treats the extracted tissue with a treating solution including one or more anionic detergents, to produce a treated tissue. The treated tissue is washed with a decontaminating solution to produce the acellular soft tissue graft; and the acellular soft tissue graft is then stored in a storage solution comprising one or more decontaminating agents.

The soft tissue process of the '018 patent includes the steps of: isolating from a suitable donor a desired tissue sample of the biological material; extracting the tissue with mildly alkaline hypotonic buffered solution of an endonuclease such as Benzonase® and a nonionic detergent formulation such as Allowash Solution™, optionally treating the tissue with a hypertonic buffered salt solution; extracting and treating the tissue with a mildly alkaline hypotonic buffered solution of sodium dodecylsulfate, optionally with 0.1 to 0.5 M sodium chloride rendering the solution hypertonic; washing the tissue with ultrapure water followed by a water solution of chloride dioxide; and storage in a sealed container in isotonic saline, chloride dioxide or 70% isopropanol.

It can thus be seen that the prior art processes require extensive chemical treatment with a multitude of process steps in an attempt to obtain an acellular sterilized soft tissue specimen which has limited shelf life.

### SUMMARY OF THE INVENTION

The present invention is a process for preparing soft tissue for implant in a human and removes cellular components from tissue taken from a mammal while sterilizing the tissue under vacuum while treating the same with an oxide or an allotropic form of oxygen. The process comprises the following steps:

(1) obtaining soft tissue from a mammal such as a human donor;

(2) processing and decellularizing the soft tissue by soaking the tissue in sodium chloride and a detergent and rinsing same with sterile water to substantially remove the residual sodium chloride and detergent;

(3) sterilizing the tissue under vacuum by treating the same with an oxide or allotropic form of oxygen taken from a group consisting of Ozone (TSO₃), Vapor H₂O₂ (VHP), Plasma H₂O₂, Ethylene Oxide Gas, and Sodium Hydroxide (NaOH);

(4) processing the tissue by cutting the tissue to size; and

(5) packaging the tissue.

It is thus an object of the invention to provide acellular allograft dermis for implantation into a human being.

It is another object of the invention to provide acellular soft tissue which is sterilized for usage as an implant by a surgeon.

It is still another object of the invention to provide acellular sterilized dermis which can be stored for long periods of time for later use by a surgeon for implantation into a human being.

These and other objects, advantages, and novel features of the present invention will become apparent when considered with the teachings contained in the detailed disclosure along with the accompanying drawings.

### BRIEF DESCRIPTION OF THE DRAWINGS

FIG. 1 is a schematic flow chart showing the soft tissue decellularization and sterilization process.

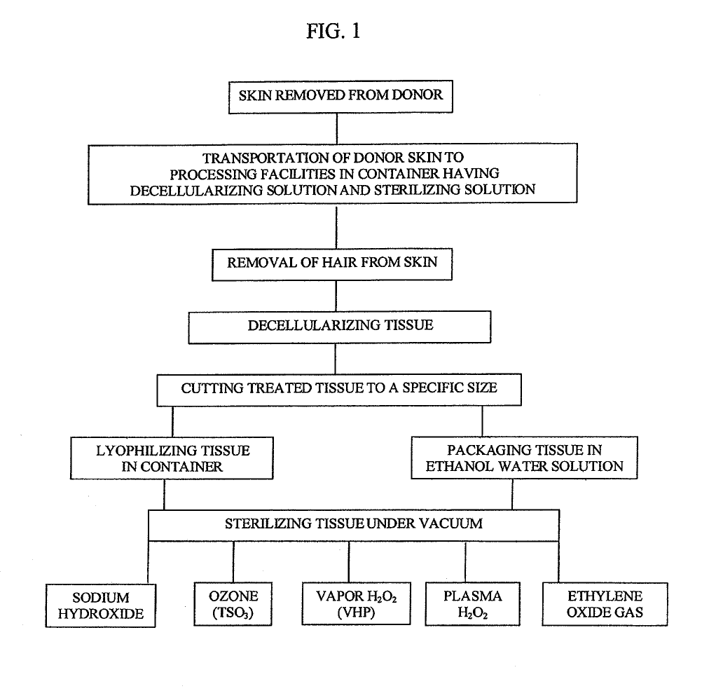

### DESCRIPTION OF THE INVENTION

The present invention is directed towards the preparation of soft tissue from a mammal, preferably from a human which is processed, decellularized and sterilized.

The preferred form of soft tissue is skin although other forms of soft tissue can be treated.

The soft tissue which is envisioned as being processed is full thickness skin which includes the epidermis, dermis and subcutaneous layers.

For the purpose of this application, the epidermis is the outer most layer of the skin and dermis is the layer of skin lying immediately under the epidermis and the term skin may refer to either epidermis, dermis or subcutaneous layers or all of the same, depending on the usage.

The skin which has been previously obtained from a donor who is deceased or living is shipped from the donor site in a container which may contain antibiotics, alcohol or mixtures of same, mixed with a decellularizing solution such as Sodium Chloride and is then frozen. This minimizes or prevents contamination of the tissue and begins the epidermal separation from the dermal skin layer. The frozen skin is then taken from the freezer and thawed in a basin filled with sterile purified water. Prior to processing, tissue is inspected for damage (holes or tears) and distinctive features (moles, warts, tattoos) which are removed using a scalpel. The tissue is inspected for hair and the same is removed using anyone of a number of techniques including chemical removal using compositions such as (1) water, mineral oil, calcium thioglycolate, calcium hydroxide, ceteareth-20, sodium hydroxide, camellia oleifera extract, sunflower seed oil, fragrance, chromium hydroxide green and alkaline soap and physical removal such as (2) hot wax, hair inhibition, non-heating type laser hair removal in ultra short pulse (USP) range and microdermabrasion. A visual inspection is performed to ensure the skin tissue has uniform thickness. Thickness is recorded using a thickness gauge. To identify the orientation (dermal or epidermal side) of tissue such as skin, the skin is positioned such that the epidermis faces the processor and an incision is cut into the upper left corner of each piece of tissue to indicate the epidermal side.

Prior to processing, the tissue form is inspected for visual defects and then trimmed for further processing.

In processing, the epidermal layer is removed and the dermis is decellularized using Sodium Chloride (NaCl) solution at a concentration of 0.1-10M, preferably about 1M with a pH ranging from 5.0-9.0, preferably 6.8-7.2, and is agitated at a speed of 65 rpm on an orbital shaker for 1-96 hours, preferably 12 hours to a maximum of 48 hours. After 12 hours, the container holding the skin is checked to ascertain if the epidermal layers have been sloughed off. If not, the container is checked every 2 hours. The dermis is then removed and placed on a cutting board with the epidermal side up and any remaining epidermal layers are picked off and discarded as well as any remaining hairs. The remaining dermis pieces are replaced in the tissue flasks, filled with sterile water and agitated on the orbital shaker for 15 minutes. The sterile water is refreshed and the rinse procedure is repeated one more time for a total of two rinses.

Once the final rinse is complete, the dermis pieces are trimmed into shaped pieces, preferably rectangular, by removing all of the rough edges of each piece with a scalpel. The trimmed dermis pieces are then immersed in 0.1% Triton X-100 solution having a concentration of 0.01-10.0%, preferably about 0.1% with a pH ranging from 4.5-8.5, preferably 6.2-7.0 and agitated on the orbital shaker for 1-96 hours, preferably 24 hours to 48 hours. The dermis is then placed in tissue flasks filled with sterile water, and agitated on the orbital shaker at 65 rpm for 15 minutes. The sterile water is refreshed and the rinse procedure is repeated a minimum of 5 more times for a total of 6 water rinses. A residual detergent test is performed on the rinsate after the 6^{th} water rinse to ensure the detergent has been adequately removed.

The treated soft tissue in the nature of acellular dermis is sterilized under vacuum with an allotropic form of oxygen such as Ozone (TSO₃) at a temperature ranging from 10° C.-50° C. for a period ranging from about 15 minutes to about 120 minutes to achieve a dosage concentration of about 5% to about 15% or by using various oxides taken from a group of Vapor H₂O₂ (VHP) at a temperature ranging from 20° C.-50° C. for a period ranging from about 30 minutes to about 180 minutes; Plasma H₂O₂ at a temperature ranging from 30° C.-60° C. for a period ranging from 30-180 minutes; Ethylene Oxide Gas at a temperature ranging from about 35° C. to about 70° C. for period ranging from about 12 hours to about 20 hours to achieve a dosage concentration of 100-1000 ppm or by using Sodium Hydroxide (NaOH) at a temperature of 10° C. 30° C. for a period ranging from about 0.5 hours to about 12 hours to achieve a dosage concentration ranging from 0.01-10M.

### Example 1

### Sterilization of Dermis under Vacuum using Ozone

The tissue which has been previously obtained from a donor is shipped from the donor site in a container having a sterilization solution mixed with a decellularizing solution such as sodium chloride and then frozen.

The donor tissue is then thawed and then rinsed to maintain moisture. The thawed tissue is processed by removing hair and is then decellularized using 1M NaCl and 0.1% of Triton X-100. If desired at the time of decellularization one or more of the following protease inhibitors may be added; Aminoethylbenzenesulfonyl fluoride HCL (serine proteases) (25-100 μm, Aprotinin (broad spectrum, serine proteases) (7.5-30 μm), Protease Inhibitor E-64 (cysteine proteases) (0.05-.0.20), Leupeptin, Hemisulfate (cysteine proteases) (0.05-.0.20 μm), EDTA, Disodium (0.025-.0.10 μm), and trypsin-like proteases, Pepstatin A (Aspartic Proteases), Marmistat (MMP2). The tissue is processed and decellularized and is inspected for visual defects and trimmed.

Once all blood and lipids are removed from the skin, the water is changed with clean sterile water. Impurities are removed from each piece of skin with a scalpel (epidermal side up during this process). Each skin piece is placed with the epidermal side up on the cutting board or flat surface and the skin is checked for damage (holes and initial tearing) and for distinctive features (mole, warts, tattoos) and these impurities are cut off using a scalpel.

Each skin piece is checked for hairs and the hairs are removed chemically by application of chemical compositions such as water, mineral oil, calcium thioglycolate, calcium hydroxide, ceteareth-20, sodium hydroxide, camellia oleifera extract, sunflower seed oil, fragrance, chromium hydroxide green after which the skin is rinsed with water. The skin is positioned with the dermis side up (epidermis down) on the cutting board and rectangular skin pieces are cut by removing the rough edges of each piece with one or more uninterrupted cuts using a scalpel and ruler. An incision is cut into the left hand corner of each piece of skin indicating the epidermal side of the skin. A visual inspection is performed to make sure the tissue has a uniform thickness throughout the piece and regions with a visibly low or non-uniform thickness are removed. A thickness measurement is then performed using a thickness gauge.

The skin is decellularized in a sterile tissue culture bottle filled with 1 L of 1M NaCl. The bottle is sealed in a self-seal pouch and the bottle is placed on its flat side on the shaker with a set speed of 65 rpm for a period of 12-48 hours. The bottle(s) is checked after the first 12 hours to see if the epidermal layers have sloughed off. After the first 12 hour check, the bottle is checked every 2 hours until all epidermal layers have been sloughed. The bottles are removed from the shaker and the NaCl is emptied from the bottle(s). The skin is removed from the bottle and placed on the cutting board with the epidermal side up. The epidermal layers are peeled off with forceps and discarded leaving only the dermal layer (dermis). The bottles are rinsed with sterile water and the peeled skin pieces (dermis) are placed back into the bottle. The bottles are then filled with enough sterile water to submerge the tissue while the bottle is lying flat and the bottle is placed on the shaker which has a preset speed of 65 rpm. The shaker is set to run for 15 minutes. After running 15 minutes, the bottle(s) are removed and the water is changed with clean sterile water. This rinse is repeated one more time for a total of two times. The bottle(s), are removed from the shaker, emptied and filled with 1 L of 0.1% Triton X-100. The bottle containing the dermis is seated in a self-seal pouch and placed on the shaker set to the speed to 65 rpm's and allowed to shake for 24 to 48 hours. The shaker is stopped after 24 hours or a later time period, the dermis is removed from the bottles and place submerged in a container with sterile water to rinse off the Triton X-100. The tissue is again rinsed with a sterile water for 15 minutes at 65 rpm's for irrigation to rinse off the Triton X-100. The rinse is repeated 5 more times for a total of 6 times. After rinsing a residual detergent test is performed to make sure that the detergent has been removed from the tissue so that less than 1 ppm is found on the tissue.

The tissue may be lyophilized or is immersed in 70% ethanol and 30% water and packaged for storage in sterile foil.

If the dermis is to be lyophilized the skin is laid flat on screens and placed in a double. Tyvek® pouch. The tissue is placed in a freezer at −70° on the lyophilization staging shelf to prevent the tissue from becoming wrinkled or deformed until the lyophilizer is available.

Upon removal from the lyophilization, the dermis tissue is cut to size and may be perforated with the perforations 10 spaced 2-3 mm apart with each perforation preferably having a diameter of about 1.2 mm.

The dermis is then sterilized under vacuum with Ozone (TSO₃) at a preferred temperature ranging from 30° C.-35° C. for a preferred period of about 1 hour to achieve a concentration of about 6% to about 12%. After treatment the dermis is sterile.

### Example 2

### Sterilization of Dermis under Vacuum with Vapor H₂O₂

The tissue which has been previously obtained from a donor is shipped from the donor site in a container having a sterilization solution mixed with a decellularizing solution such as sodium chloride and then frozen.

The donor tissue is then thawed and then rinsed to maintain moisture. The thawed tissue is processed by removing hair and is then decellularized using 1M NaCl and 0.1% of Triton X-100. If desired at the time of decellularization one or more of the following protease inhibitors may be added; Aminoethylbenzenesulfonyl fluoride HCL (serine proteases) (25-100 Aprotinin (broad spectrum, serine proteases) (7.5-30 μm), Protease Inhibitor E-64 (cysteine proteases) (0.05-.0.20 μm), Leupeptin, Hemisulfate (cysteine proteases) (0.05-.0.20 μm), EDTA, Disodium (0.025-.0.10 μm), and trypsin-like proteases, Pepstatin A (Aspartic Proteases), Marmistat (MMP2). The tissue is processed and decellularized and is inspected for visual defects and trimmed.

Once all blood and lipids are removed from the skin, the water is changed with clean sterile water. Impurities are removed from each piece of skin with a scalpel (epidermal side up during this process). Each skin piece is placed with the epidermal side up on the cutting board or flat surface and the skin is checked for damage (holes and initial tearing) and for distinctive features (mole, warts, tattoos) and these impurities are cut off using a scalpel.

Each skin piece is checked for hairs and the hairs are removed chemically by application of chemical compositions such as water, mineral oil, calcium thioglycolate, calcium hydroxide, ceteareth-20, sodium hydroxide, camellia oleifera extract, sunflower seed oil, fragrance, chromium hydroxide green after which the skin is rinsed with water. The skin is positioned with the dermis side up (epidermis down) on the cutting board and rectangular skin pieces are cut by removing the rough edges of each piece with one or more uninterrupted cuts using a scalpel and ruler. An incision is cut into the left hand corner of each piece of skin indicating the epidermal side of the skin. A visual inspection is performed to make sure the tissue has a uniform thickness throughout the piece and regions with a visibly low or non-uniform thickness are removed. A thickness measurement is then performed using a thickness gauge.

The skin is decellularized in a sterile tissue culture bottle filled with 1 L of 1M NaCl. The bottle is sealed in a self-seal pouch and the bottle is placed on its flat side on the shaker with a set speed of 65 rpm for a period of 12-48 hours. The bottle(s) is checked after the first 12 hours to see if the epidermal layers have sloughed off. After the first 12 hour check, the bottle is checked every 2 hours until all epidermal layers have been sloughed. The bottles are removed from the shaker and the NaCl is emptied from the bottle(s). The skin is removed from the bottle and placed on the cutting board with the epidermal side up. The epidermal layers are peeled off with forceps and discarded leaving only the dermal layer (dermis). The bottles are rinsed with sterile water and the peeled skin pieces (dermis) are placed back into the bottle. The bottles are then filled with enough sterile water to submerge the tissue while the bottle is lying flat and the bottle is placed on the shaker which has a preset speed of 65 rpm. The shaker is set to run for 15 minutes. After running 15 minutes, the bottle(s) are removed and the water is changed with clean sterile water. This rinse is repeated one more time for a total of two times. The bottle(s) are removed from the shaker, emptied and filled with 1 L of 0.1% Triton X-100. The bottle containing the dermis is seated in a self-seal pouch and placed on the shaker set to the speed to 65 rpm's and allowed to shake for 24 to 48 hours. The shaker is stopped after 24 hours or a later time period, the dermis is removed from the bottles and place submerged in a container with sterile water to rinse off the Triton X-100. The tissue is again rinsed with a sterile water for 15 minutes at 65 rpm's for irrigation to rinse off the Triton X-100. The rinse is repeated 5 more times for a total of 6 times. After rinsing a residual detergent test is performed to make sure that the detergent has been removed from the tissue so that less than 1 ppm is found on the tissue.

The tissue may be lyophilized or is immersed in 70% ethanol and 30% water and packaged for storage in sterile foil.

If the dermis is to be lyophilized the skin is laid flat on screens and placed in a double Tyvek® pouch. The tissue is placed in a freezer at −70° on the lyophilization staging shelf to prevent the tissue from becoming wrinkled or deformed until the lyophilizer is available.

Upon removal from the lyophilization, the dermis tissue is cut to size and may be perforated with the perforations 10 spaced 2-3 mm apart with each perforation preferably having a diameter of about 1.2 mm.

The dermis is then sterilized under vacuum with Vapor H₂O₂ (VHP) at a preferred temperature ranging from about 30° C. to about 40° C. for a preferred period ranging from about 0.5 hours to about 3 hours. After treatment the dermis is sterile.

### Example 3

### Sterilization of Dennis under Vacuum with Plasma H₂O₂

The tissue which has been previously obtained from a donor is shipped from the donor site in a container having a sterilization solution mixed with a decellularizing solution such as sodium chloride and then frozen.

The donor tissue is then thawed and then rinsed to maintain moisture. The thawed tissue is processed by removing hair and is then decellularized using 1M NaCl and 0.1% of Triton X-100. If desired at the time of decellularization one or more of the following protease inhibitors may be added; Aminoethylbenzenesulfonyl fluoride HCL (serine proteases) (25-100 μm, Aprotinin (broad spectrum, serine proteases) (7.5-30 μm), Protease Inhibitor E-64 (cysteine proteases) (0.05-.0.20 μm), Leupeptin, Hemisulfate (cysteine proteases) (0.05-.0.20 μm), EDTA, Disodium (0.025-.0.10 μm), and trypsin-like proteases, Pepstatin A (Aspartic Proteases), Marmistat (MMP2). The tissue is processed and decellularized and is inspected for visual defects and trimmed.

Once all blood and lipids are removed from the skin, the water is changed with clean sterile water. Impurities are removed from each piece of skin with a scalpel (epidermal side up during this process). Each skin piece is placed with the epidermal side up on the cutting board or flat surface and the skin is checked for damage (holes and initial tearing) and for distinctive features (mole, warts, tattoos) and these impurities are cut off using a scalpel.

Each skin piece is checked for hairs and the hairs are removed chemically by application of chemical compositions such as water, mineral oil, calcium thioglycolate, calcium hydroxide, ceteareth-20, sodium hydroxide, camellia oleifera extract, sunflower seed oil, fragrance, chromium hydroxide green after which the skin is rinsed with water. The skin is positioned with the dermis side up (epidermis down) on the cutting board and rectangular skin pieces are cut by removing the rough edges of each piece with one or more uninterrupted cuts using a scalpel and ruler. An incision is cut into the left hand corner of each piece of skin indicating the epidermal side of the skin. A visual inspection is performed to make sure the tissue has a uniform thickness throughout the piece and regions with a visibly low or non-uniform thickness are removed. A thickness measurement is then performed using a thickness gauge.

The skin is decellularized in a sterile tissue culture bottle filled with 1 L of 1M NaCl. The bottle is sealed in a self-seal pouch and the bottle is placed on its flat side on the shaker with a set speed of 65 rpm for a period of 12-48 hours. The bottle(s) is checked after the first 12 hours to see if the epidermal layers have sloughed off. After the first 12 hour check, the bottle is checked every 2 hours until all epidermal layers have been sloughed. The bottles are removed from the shaker and the NaCl is emptied from the bottle(s). The skin is removed from the bottle and placed on the cutting board with the epidermal side up. The epidermal layers are peeled off with forceps and discarded leaving only the dermal layer (dermis). The bottles are rinsed with sterile water and the peeled skin pieces (dermis) are placed back into the bottle. The bottles are then filled with enough sterile water to submerge the tissue while the bottle is lying flat and the bottle is placed on the shaker which has a preset speed of 65 rpm. The shaker is set to run for 15 minutes. After running 15 minutes, the bottle(s) are removed and the water is changed with clean sterile water. This rinse is repeated one more time for a total of two times. The bottle(s) are removed from the shaker, emptied and filled with 1 L of 0.1% Triton X-100. The bottle containing the dermis is seated in a self-seal pouch and placed on the shaker set to the speed to 65 rpm's and allowed to shake for 24 to 48 hours. The shaker is stopped after 24 hours or a later time period, the dermis is removed from the bottles and place submerged in a container with sterile water to rinse off the Triton X-100. The tissue is again rinsed with a sterile water for 15 minutes at 65 rpm's for irrigation to rinse off the Triton X-100. The rinse is repeated 5 more times for a total of 6 times. After rinsing a residual detergent test is performed to make sure that the detergent has been removed from the tissue so that less than 1 ppm is found on the tissue.

The tissue may be lyophilized or is immersed in 70% ethanol and 30% water and packaged for storage in sterile foil.

If the dermis is to be lyophilized the skin is laid flat on screens and placed in a double Tyvek® pouch. The tissue is placed in a freezer at −70° on the lyophilization staging shelf to prevent the tissue from becoming wrinkled or deformed until the lyophilizer is available.

Upon removal from the lyophilization, the dermis tissue is cut to size and may be perforated with the perforations 10 spaced 2-3 mm apart with each perforation preferably having a diameter of about 1.2 mm.

The dermis is then treated under vacuum with Plasma H₂O₂ at a preferred temperature ranging from about 45° C. to about 50° C. for a preferred period ranging from about 55 minutes to about 70 minutes. After treatment the dermis is sterile.

### Example 4

### Sterilization of Dermis under Vacuum with Ethylene Oxide

The tissue which has been previously obtained from a donor is shipped from the donor site in a container having a sterilization solution mixed with a decellularizing solution such as sodium chloride and then frozen.

The donor tissue is then thawed and then rinsed to maintain moisture. The thawed tissue is processed by removing hair and is then decellularized using 1M NaCl and 0.1% of Triton X-100. If desired at the time of decellularization one or more of the following protease inhibitors may be added; Aminoethylbenzenesulfonyl fluoride HCL (serine proteases) (25-100 μm Aprotinin (broad spectrum, serine proteases) (7.5-3 μm), Protease Inhibitor E-64 (cysteine proteases) (0.05-.0.20 μm), Leupeptin, Hemisulfate (cysteine proteases) (0.05-.0.20 μm), EDTA, Disodium (0.025-.0.1 μm), and trypsin-like proteases, Pepstatin A (Aspartic Proteases), Marmistat (MMP2). The tissue is processed and decellularized and is inspected for visual defects and trimmed.

Once all blood and lipids are removed from the skin, the water is changed with clean sterile water. Impurities are removed from each piece of skin with a scalpel (epidermal side up during this process). Each skin piece is placed with the epidermal side up on the cutting board or flat surface and the skin is checked for damage (holes and initial tearing) and for distinctive features (mole, warts, tattoos) and these impurities are cut off using a scalpel.

Each skin piece is checked for hairs and the hairs are removed chemically by application of chemical compositions such as water, mineral oil, calcium thioglycolate, calcium hydroxide, ceteareth-20, sodium hydroxide, camellia oleifera extract, sunflower seed oil, fragrance, chromium hydroxide green after which the skin is rinsed with water. The skin is positioned with the dermis side up (epidermis down) on the cutting board and rectangular skin pieces are cut by removing the rough edges of each piece with one or more uninterrupted cuts using a scalpel and ruler. An incision is cut into the left hand corner of each piece of skin indicating the epidermal side of the skin. A visual inspection is performed to make sure the tissue has a uniform thickness throughout the piece and regions with a visibly low or non-uniform thickness are removed. A thickness measurement is then performed using a thickness gauge.

The skin is decellularized in a sterile tissue culture bottle filled with IL of 1M NaCl. The bottle is sealed in a self-seal pouch and the bottle is placed on its flat side on the shaker with a set speed of 65 rpm for a period of 12-48 hours. The bottle(s) is checked after the first 12 hours to see if the epidermal layers have sloughed off. After the first 12 hour check, the bottle is checked every 2 hours until all epidermal layers have been sloughed. The bottles are removed from the shaker and the NaCl is emptied from the bottle(s). The skin is removed from the bottle and placed on the cutting board with the epidermal side up. The epidermal layers are peeled off with forceps and discarded leaving only the dermal layer (dermis). The bottles are rinsed with sterile water and the peeled skin pieces (dermis) are placed back into the bottle. The bottles are then filled with enough sterile water to submerge the tissue while the bottle is lying flat and the bottle is placed on the shaker which has a preset speed of 65 rpm. The shaker is set to run for 15 minutes. After running 15 minutes, the bottle(s) are removed and the water is changed with clean sterile water. This rinse is repeated one more time for a total of two times. The bottle(s) are removed from the shaker, emptied and filled with 1 L of 0.1% Triton X-100. The bottle containing the dermis is seated in a self-seal pouch and placed on the shaker set to the speed to 65 rpm's and allowed to shake for 24 to 48 hours. The shaker is stopped after 24 hours or a later time period, the dermis is removed from the bottles and place submerged in a container with sterile water to rinse off the Triton X-100. The tissue is again rinsed with a sterile water for 15 minutes at 65 rpm's for irrigation to rinse off the Triton X-100. The rinse is repeated 5 more times for a total of 6 times. After rinsing a residual detergent test is performed to make sure that the detergent has been removed from the tissue so that less than 1 ppm is found on the tissue.

The tissue may be lyophilized or is immersed in 70% ethanol and 30% water and packaged for storage in sterile foil.

If the dermis is to be lyophilized the skin is laid flat on screens and placed in a double Tyvek® pouch. The tissue is placed in a freezer at −70° on the lyophilization staging shelf to prevent the tissue from becoming wrinkled or deformed until the lyophilizer is available.

Upon removal from the lyophilization, the dermis tissue is cut to size and may be perforated with the perforations 10 spaced 2-3 mm apart with each perforation preferably having a diameter of about 1.2 mm.

The dermis is then treated under vacuum with Ethylene Oxide Gas at a preferred temperature ranging from about 50° C. to about 60° C. for a preferred period ranging from about 16 to about 18 hours to achieve a dose or concentration of about 100-1000 ppm. After treatment the dermis is sterile.

### Example 5

### Sterilization of Dermis under Vacuum with Sodium Hydroxide

The tissue which has been previously obtained from a donor is shipped from the donor site in a container sterilization solution mixed with a decellularizing solution such as sodium chloride and then frozen.

The donor tissue is then thawed and then rinsed to maintain moisture. The thawed tissue is processed by removing hair and is then decellularized using 1M NaCl and 0.1% of Triton X-100. If desired at the time of decellularization one or more of the following protease inhibitors may be added; Aminoethylbenzenesulfonyl fluoride HCL (serine proteases) (25-100 μm, Aprotinin (broad spectrum, serine proteases) (7.5-30 μm), Protease Inhibitor E-64 (cysteine proteases) (0.05-.0.20 μm), Leupeptin, Hemisulfate (cysteine proteases) (0.05-.0.20 μm), EDTA, Disodium (0.025-.0.10 μm), and trypsin-like proteases, Pepstatin A (Aspartic Proteases), Marmistat (MMP2). The tissue is processed and decellularized and is inspected for visual defects and trimmed.

Once all blood and lipids are removed from the skin, the water is changed with clean sterile water. Impurities are removed from each piece of skin with a scalpel (epidermal side up during this process). Each skin piece is placed with the epidermal side up on the cutting board or flat surface and the skin is checked for damage (holes and initial tearing) and for distinctive features (mole, warts, tattoos) and these impurities are cut off using a scalpel.

Each skin piece is checked for hairs and the hairs are removed chemically by application of chemical compositions such as water, mineral oil, calcium thioglycolate, calcium hydroxide, ceteareth-20, sodium hydroxide, camellia oleifera extract, sunflower seed oil, fragrance, chromium hydroxide green after which the skin is rinsed with water. The skin is positioned with the dermis side up (epidermis down) on the cutting board and rectangular skin pieces are cut by removing the rough edges of each piece with one or more uninterrupted cuts using a scalpel and ruler. An incision is cut into the left hand corner of each piece of skin indicating the epidermal side of the skin. A visual inspection is performed to make sure the tissue has a uniform thickness throughout the piece and regions with a visibly low or non-uniform thickness are removed. A thickness measurement is then performed using a thickness gauge. The skin is decellularized in a sterile tissue culture bottle filled with 1 L of 1M NaCl. The bottle is sealed in a self-seal pouch and the bottle is placed on its flat side on the shaker with a set speed of 65 rpm for a period of 12-48 hours. The bottle(s) is checked after the first 12 hours to see if the epidermal layers have sloughed off. After the first 12 hour check, the bottle is checked every 2 hours until all epidermal layers have been sloughed. The bottles are removed from the shaker and the NaCl is emptied from the bottle(s). The skin is removed from the bottle and placed on the cutting board with the epidermal side up. The epidermal layers are peeled off with forceps and discarded leaving only the dermal layer (dermis). The bottles are rinsed with sterile water and the peeled skin pieces (dermis) are placed back into the bottle. The bottles are then filled with enough sterile water to submerge the tissue while the bottle is lying flat and the bottle is placed on the shaker which has a preset speed of 65 rpm. The shaker is set to run for 15 minutes. After running 15 minutes, the bottle(s) are removed and the water is changed with clean sterile water. This rinse is repeated one more time for a total of two times. The bottle(s) are removed from the shaker, emptied and filled with 1 L of 0.1% Triton X-100. The bottle containing the dermis is seated in a self-seal pouch and placed on the shaker set to the speed to 65 rpm's and allowed to shake for 24 to 48 hours. The shaker is stopped after 24 hours or a later time period, the dermis is removed from the bottles and place submerged in a container with sterile water to rinse off the Triton X-100. The tissue is again rinsed with a sterile water for 15 minutes at 65 rpm's for irrigation to rinse off the Triton X-100. The rinse is repeated 5 more times for a total of 6 times. After rinsing a residual detergent test is performed to make sure that the detergent has been removed from the tissue so that less than 1 ppm is found on the tissue.

The tissue may be lyophilized or is immersed in 70% ethanol and 30% water and packaged for storage in sterile foil.

If the dermis is to be lyophilized the skin is laid flat on screens and placed in a double Tyvek® pouch. The tissue is placed in a freezer at −70° on the lyophilization staging shelf to prevent the tissue from becoming wrinkled or deformed until the lyophilizer is available.

Upon removal from the lyophilization, the dermis tissue is cut to size and may be perforated with the perforations 10 spaced 2-3 mm apart with each perforation preferably having a diameter of about 1.2 mm.

The dermis is then treated under vacuum with Sodium Hydroxide (NaOH) at a preferred temperature ranging from 10° C. to about 30° C. for a preferred period ranging from about 0.5 to about 12 hours to achieve a concentration of about 1M. After treatment the dermis is sterile.

While specific embodiments of the subject invention have been discussed, the above specification is illustrative and not restrictive. One skilled in the art will appreciate that numerous changes and modifications can be made to the invention, and that such changes and modifications can be made without departing from the spirit and scope of the invention. The full scope of the invention should be determined by reference to the claims, along with their full scope of equivalents, and the specification, along with such variations.

Each patent, patent application, and publication cited or described in the present application is hereby incorporated by reference in its entirety as if each individual patent, patent application, or publication was specifically and individually indicated to be incorporated by reference.

What is claimed is:

1. A method for the treatment of soft tissue obtained from a mammal to prepare the same for implantation into a human comprising the steps of
(a) decellularizing the soft tissue obtained from a mammal;
(b) sterilizing the soft tissue by subjecting the same to ozone under vacuum at a temperature ranging from about 10° C. to about 50° C. for a period ranging from about 0.25 to about 2 hours to achieve a concentration of about 5% to about 15%;

2. The method as claimed in claim 1 wherein said soft tissue is human skin.

3. The method as claimed in claim 2 wherein after step (a) there is an additional step of removing an epidermal layer from the skin.

4. The method as claimed in claim 2 wherein said human skin is dermis.

5. The method as claimed in claim 1 wherein said soft tissue is human tissue.

6. A method for the treatment of skin obtained from a human donor to prepare the same for implantation into a human comprising the steps of
(a) decellularizing the skin obtained from said human donor;
(b) sterilizing the skin by subjecting the same under vacuum with Ozone at a temperature ranging from about 30° C. to about 35° C. for a period of about 1 hour to achieve a concentration of about 6% to about 12%.

7. A method for the treatment of skin as claimed in claim 6 where said skin is dermis.

8. The method as claimed in claim 6 wherein after step (a) there is an additional step of removing an epidermal layer from the skin.

9. A method for the treatment of soft tissue obtained from a mammal to prepare the same for implantation into a human comprising the steps of:
(a) decellularizing the soft tissue obtained from said mammal;
(b) sterilizing the soft tissue by subjecting the same under vacuum with vapor H₂O₂ at a temperature ranging from 20° C.-50° C. for a period ranging from about 0.5 to about 3 hours.

10. The method as claimed in claim 9 wherein said soft tissue is human skin.

11. The method as claimed in claim 10 wherein after step (a) there is an additional step of removing an epidermal layer from the skin.

12. The method as claimed in claim 10 wherein said skin is dermis.

13. The method as claimed in claim 9 wherein said soft tissue is human tissue.

14. The method as claimed in claim 9 wherein said temperature ranges from about 30° C. to about 40° C. and said soft tissue is obtained from a human donor.

15. The method as claimed in claim 14 wherein said soft tissue is dermis.

16. A method for the treatment of soft tissue obtained from a mammal to prepare the same for implantation into a human comprising the steps of:
(a) decellularizing the soft tissue obtained from a mammal;
(b) sterilizing the soft tissue by subjecting the same to plasma H₂O₂ under vacuum at a temperature ranging from about 30° C. to about 60° C. for a period ranging from about 0.5 to about 3 hours.

17. The method as claimed in claim 16 wherein said soft tissue is human skin.

18. The method as claimed in claim 17 wherein after step (a) there is an additional step of removing an epidermal layer from the skin.

19. The method as claimed in claim 17 wherein said human skin is dermis.

20. The method as claimed in claim 16 wherein said soft tissue is human tissue.

21. A method for the treatment of skin obtained from a human donor to prepare the same for implantation into a human comprising the steps of:
(a) decellularizing the skin obtained from said human donor;
(b) sterilizing the skin by subjecting the same to with plasma H₂O₂ at a temperature ranging from 45° C. to about 50° C. for a period ranging from about 55 to about 70 minutes.

22. A method for the treatment of skin as claimed in claim 21 wherein said skin is dermis.

23. The method as claimed in claim 21 wherein after step (a) there is an additional step of removing an epidermal layer from the skin.

24. A method for the treatment of soft tissue obtained from a mammal to prepare the same for implantation into a human comprising the steps of:
(a) decellularizing the soft tissue obtained from a mammal;
(b) sterilizing the soft tissue by subjecting the same to ethylene oxide gas under vacuum at a temperature ranging from about 35° C. to about 70° C. for a period ranging from about 12 to about 20 hours to achieve a concentration of about 100 to about 1000 ppm.

25. The method as claimed in claim 24 wherein said soft tissue is human skin.

26. The method as claimed in claim 25 wherein after step (a) there is an additional step of removing an epidermal layer from the skin.

27. The method as claimed in claim 25 wherein said human skin is dermis.

28. The method as claimed in claim 24 wherein said soft tissue is human tissue.

29. A method for the treatment of skin obtained from a human donor to prepare the same for implantation into a human comprising the steps of:
(a) decellularizing the skin obtained from said human donor;
(b) sterilizing the skin by subjecting the same under vacuum with ethylene oxide Gas at a temperature ranging from about 50° C. to about 60° C. for a period of about 16 hours to about 18 hours to achieve a concentration of about 100-1000 ppm.

30. A method for the treatment of skin as claimed in claim 29 where said skin is dermis.

31. The method as claimed in claim 29 wherein after step (a) there is an additional step of removing an epidermal layer from the skin.

32. A method for the treatment of soft tissue obtained from a mammal to prepare the same for implantation into a human comprising the steps of:
(a) decellularizing the soft tissue obtained from a mammal;
(b) sterilizing the soft tissue by subjecting the same to sodium hydroxide under vacuum at a temperature ranging from about 10° C. to about 30° C. for a period ranging from about 0.5 hours to about 12 hours to achieve a concentration of about 0.01 to about 10M.

33. The method as claimed in claim 32 wherein said soft tissue is human skin.

34. The method as claimed in claim 33 wherein after step (a) there is an additional step of removing an epidermal layer from the skin.

35. The method as claimed in claim 33 wherein said human skin is dermis.

36. The method as claimed in claim 32 wherein said soft tissue is human tissue.

37. A method for the treatment of skin obtained from a human donor to prepare the same for implantation into a human comprising the steps of
(a) decellularizing the skin obtained from said human donor;
(b) sterilizing the skin by subjecting the same under vacuum with sodium hydroxide at a temperature ranging from about 10° C. to about 30° C. for a period of about 0.5 hour to about 12 hours to achieve a concentration of about 1M.

38. A method for the treatment of skin as claimed in claim 37 where said skin is dermis.

39. The method as claimed in claim 37 wherein after step (a) there is an additional step of removing an epidermal layer from the skin.
